(19) United States
(12) Patent Application Publication
(10) Pub. No.: US 2013/0190682 A1
(43) Pub. Date: Jul. 25, 2013

(54) METHODS AND SYSTEMS FOR CONTROLLING MEDICAL ENVIRONMENTS

(71) Applicants: Peter M. Bonutti, Effingham, IL (US); Justin Beyers, Effingham, IL (US)

(72) Inventors: Peter M. Bonutti, Effingham, IL (US); Justin Beyers, Effingham, IL (US)

(21) Appl. No.: 13/559,352

(22) Filed: Jul. 26, 2012

Related U.S. Application Data
(60) Provisional application No. 61/511,649, filed on Jul. 26, 2011.

(51) Int. Cl. A61M 5/44; A61M 5/172

(52) U.S. Cl. CPC A61M 5/44; A61M 5/172; A61M 5/1723
USPC 604/66; 604/114; 604/65

(57) ABSTRACT

A system for controlling an environment of a patient undergoing an surgical procedure is provided. The system includes a conduit configured to channel fluids towards the patient, a sensor configured to measure at least one operating parameter of the fluid being channeled to the patient, an effector configured to change the operating parameter of the fluid, and a controller configured to selectively activate the effector based on a signal received from the sensor.

### CROSS-REFERENCE TO RELATED APPLICATIONS

This application claims the benefit of U.S. Provisional Patent Application 61/511,649 to the same inventor, filed Jul. 26, 2011, entitled SYSTEMS FOR CONTROLLING MEDICAL ENVIRONMENTS, the entire contents of which are incorporated herein by reference.

### FIELD

The disclosure relates to controlling, measuring, changing, and/or monitoring the temperature, pH level, moisture, pharmaceutical and/or therapeutic agent level, and/or other parameters of an environment and/or a fluid surrounding, entering, or exiting a device, system, or a body of a patient or portion thereof. Embodiments may include an effector, controller, sensor, conduit, and/or channel for measuring and/or controlling one or more parameters of a body and/or fluid.

### BACKGROUND

During a surgical procedure, many factors can affect the success of the procedure. For example changes in the body temperature and/or in the ambient environment, can affect the way in which the body responds to a procedure and/or drugs that may be administered during the procedure. Generally, the body of a human or other mammal maintains a certain level of temperature, pH, and humidity. The temperature and pH level within the body can vary throughout the body. For example, certain body parts, such as the stomach or intestines, may have a different pH level and temperature than the brain or heart. Also, temperature and pH levels vary in the body throughout the day, depending on the level of activity of a particular person. As such, a person when sleeping will generally have different pH levels than the same person when exercising. The temperature within a body can also be affected by disease, trauma, and/or injury to tissue. In such instances, the temperature of the body may fluctuate to aid in healing, to aid in fighting infections, and/or to aid in resisting or killing a foreign object.

Generally, current surgical procedures may inhibit a body's ability to efficiently heal as a surgical environment can impede and potentially inhibit a body's natural healing response to a surgical procedure. For example, using cold or chilled materials (e.g., water and/or plasma) during a procedure may slow the healing process, initiate pain in effected tissue, and/or impede drug efficacy. As such, a need exists for a device and method for controlling, measuring, changing, and/or monitoring the environment and/or a fluid surrounding, entering, and/or exiting the body of a patient.

### SUMMARY

In one embodiment, a system for controlling an environment of a patient undergoing an surgical procedure is provided. The system includes a conduit configured to channel fluids towards the patient, a sensor configured to measure at least one operating parameter of the fluid being channeled to the patient, an effector configured to change the operating parameter of the fluid, and a controller configured to selectively activate the effector based on a signal received from the sensor.

In another embodiment, a system for controlling an environment of a body portion of patient is provided. The system includes a conduit configured to channel fluid towards the patient, a sensor configured to measure an operating parameter of the fluid being channeled to the patient, a first reservoir configured to selectively release a first substance into the conduit to facilitate increasing the operating parameter of the fluid, a second reservoir configured to selectively release a second substance into the conduit to facilitate decreasing the operating parameter of the fluid, and a controller configured to control release of the first and second substances from the first and second reservoirs based on a signal received from the sensor.

In an alternative embodiment, a system for use in controlling an environment of a patient is provided. The system includes a fluid sensor configured to measure a fluid parameter of a fluid channeled towards a patient, a body sensor configured to measure a body parameter of the patient, and a controller. The controller is configured to compare at least one of the fluid parameter and body parameter to a predefined target parameter and change the fluid parameter based on the comparison. The system also includes an effector configured to change the fluid parameter in response to the signal from the controller.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 illustrates a schematic view of an environment management system 100 that may be used to manage and/or to control surgical area environments of a patient undergoing a surgical procedure.

FIG. 2 illustrates a schematic view an alternative environment management system that may be used in managine and/or controlling surgical area environments of a patient.

FIG. 3 illustrates a schematic view of an environment management system 300A with environment management system 100 shown in FIG. 1.

FIG. 4 illustrates a schematic view of an environment management system 300B with environment management system 200 shown in FIG. 2.

FIG. 5 illustrates a schematic of an exemplary environment management system that may be used to adjust and/or control one or more parameters of a body undergoing a surgical procedure.

FIG. 6 illustrates a schematic of an exemplary environment management system including the controller shown in FIG. 5 and configured for arthroscopic surgery.

FIG. 7 illustrates a schematic of an exemplary environment management system configured for laparoscopic surgery.

FIG. 8 illustrates a schematic of an exemplary environment management system including a blanket and configured to adjust and/or maintain one or more parameters of a body.

FIG. 9 illustrates a schematic of an exemplary environment management system configured for open surgery.

FIG. 10 illustrates an embodiment a schematic of an exemplary environment management system including wireless control.

### DETAILED DESCRIPTION

The present disclosure relates to devices and methods for controlling the environments of living organisms and/or the environments surrounding a surgical area. Characteristics of the environment or parameters that may be controlled include, but are not limited to only including, are temperature, pH, moisture, humidity, oxygen content and percentage, oxygen tension, oxygenase, carbon dioxide content and percentage, rate of blood flow, nutrient-content, osmolarity, pressure, vascular permeability, electrical charge, particle size, and/or the presence of pharmaceutical or therapeutic agents. Such characteristics may be measured, changed, and monitored automatically and/or selectively by a user to facilitate the optimizing of the environment adjacent to a particular body region.

Controlling the characteristics or parameters of an environment of a living organism may, for example, to promote cell function and/or facilitate the success of a medical procedure. Such characteristics or parameters may include, but are not limited to, temperature, pH level, moisture, humidity, oxygen tension, oxygenase, carbon dioxide tension, rate of blood flow, nutrient-content, osmolarity, pressure, vascular permeability, electrical charge, and the presence of pharmaceutical agents. As a result of disease, age, injury, or surgery. For example, it may beneficial to provide supplemental control to the environment of a body region. Other benefits for controlling the environment may include effecting cell receptors, effecting hormone release, effecting tissue healing, limiting the ability of bacteria to multiple, effecting virus activity, stimulating white blood cells enzyme release, stimulating white blood cell phagocytosis or migration, and/or managing pain. Moreover, optimizing the environment with the devices and methods of the present disclosure may facilitate enhancing or improving the effect of therapeutic/pharmaceutical agents, facilitate enhanced pain management and therapeutic treatments, facilitate improving the performance or outcome of a surgical procedure or intervention, facilitate enhancing the results of a surgical implant, facilitate optimizing cell or tissue ingrowth when using cell therapy or gene therapy, and provide other advantages, some of which are described here in relation to the exemplary embodiments.

Controlling the environment with this multimodal approach may be performed preoperatively, during surgical treatment, and postoperatively. By regulating the local body and/or the core body temperature, and/or by controlling the local pH level or other factors as described herein, desiccation may be minimized, and vascular flow may be promoted. In addition, oxygen tension and/or nutrient delivery to the patient may be optimized and tissue osmolarity may be maintained or selectively controlled. Moreover, in at least some embodiments, access of therapeutic substances to the environment may be controlled. Such therapeutic substances may be transcutaneously or percutaneously delivered, and may include, for example, antibiotics, hydroxypatite, anti-inflammatory agents, steroids, antibiotics, analgesic agents, chemotherapeutic agents, bone morphogenetic protein, demineralized bone matrix, collagen, growth factors, autogenetic bone marrow, progenitor cells, calcium sulfate, immu-suppressants, fibrin, osteoinductive materials, apatite compositions, fetal cells, stem cells, enzymes, proteins, hormones, germicides, non-proliferative agents, anti-coagulants, anti-platelet agents, Tyrosine Kinase inhibitors, anti-infective agents, anti-tumor agents, anti-leukemic agents, and/or combinations thereof

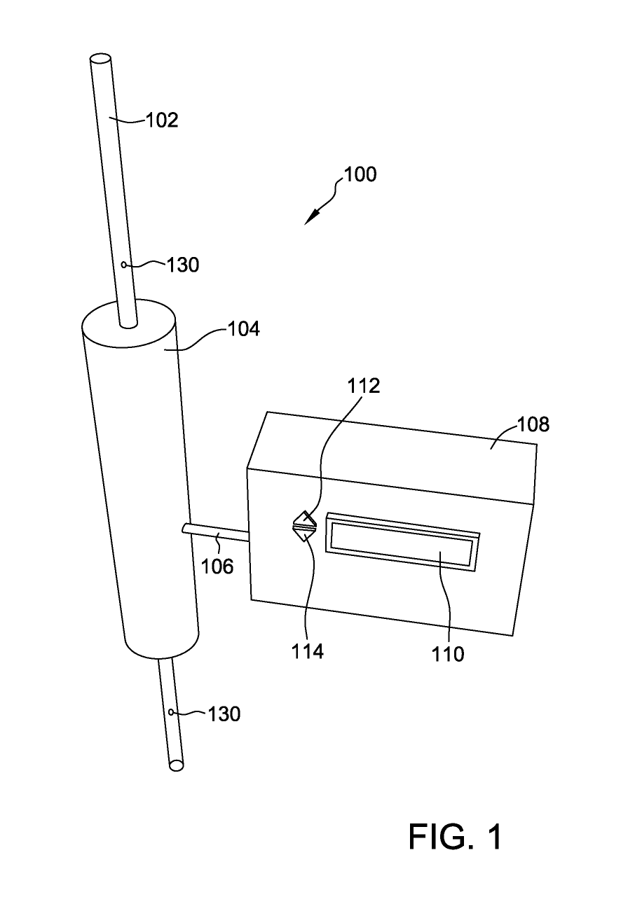

Referring to the Figures, FIG. 1 is view of an environment management system 100 that may be used to manage and/or to control surgical area environments of a patient undergoing a surgical procedure. In the exemplary embodiment, system 100 includes at least a conduit 102, an effector 104, and a computing device or controller 108. In the exemplary embodiment, computing device 8 includes memory (not shown) and a processor (not shown) that is coupled to memory for executing programmed instructions. The processor may include one or more processing units (e.g., in a multi-core configuration). Computing device 108 is programmable to perform one or more operations described herein by programming the memory and/or the processor, for example. In one embodiment, the processor may be programmed by encoding an operation as one or more executable instructions, and by providing the executable instructions in the memory.

The processor may include, but is not limited to, a general purpose central processing unit (CPU), a microcontroller, a reduced instruction set computer (RISC) processor, an application specific integrated circuit (ASIC), a programmable logic circuit (PLC), and/or any other circuit or processor capable of executing the functions described herein. The methods described herein may be encoded as executable instructions embodied in a computer-readable medium including, without limitation, a storage device and/or a memory device. Such instructions, when executed by the processor, cause the processor to perform at least a portion of the methods described herein. The above examples are exemplary only, and thus are not intended to limit in any way the definition and/or meaning of the term “processor”.

The memory, as described herein, is one or more devices that enable information such as executable instructions and/or other data to be stored and retrieved. The memory may include one or more computer-readable media, such as, without limitation, dynamic random access memory (DRAM), static random access memory (SRAM), a solid state disk, and/or a hard disk. Moreover, the memory may be configured to store, without limitation, surgical requirements, patient requirements, and/or any other type of data suitable for use with the methods and systems described herein.

In the exemplary embodiment, computing device 108 includes a presentation device 110 that is coupled to the processor. Presentation device 110 outputs by, for example, displaying, printing, and/or otherwise outputting information such as, but not limited to, documents, interfaces, warnings, videos, photos, and/or any other type of data to a user. For example, presentation device 110 may include a display adapter that is coupled to a display device, such as a cathode ray tube (CRT), a liquid crystal display (LCD), a light-emitting diode (LED) display, an organic LED (OLED) display, and/or an “electronic ink” display. In some embodiments, presentation device 110 includes more than one display device. In addition, or in the alternative, presentation device 110 may include a printer.

In the exemplary embodiment, computing device 108 includes at least one input device 112 that receives input from a user. For example, input device 112 may receive input, selections, and/or any other type of inputs from a user suitable for use with the methods and systems described herein. In the exemplary embodiment, input device 112 is coupled to the processor and may include, for example, a keyboard, a pointing device, a mouse, a stylus, a touch sensitive panel (e.g., a touch pad or a touch screen), and/or an audio input device. In the exemplary embodiment, input device 112 is a plurality of buttons that enable a user to select a desired setting.

In the exemplary embodiment, computing device 108 includes one or more communication devices 106 coupled to memory and/or the processor. Each communication device 106 is coupled in communication with a device 106 located remotely from computing device 108. In one embodiment, device 106 may be a sensor 130 or another computing device 108. For example, communication device 106 may include, without limitation, a wired network adapter, a wireless network adapter, a Bluetooth adapter, and/or a mobile telecommunications adapter. In at least one embodiment, computing device 108 includes a processor and one or more communication devices incorporated into it or integrated with the processor. It should be appreciated that communication device (or another communication device) may be separate from processor and/or engage processor.

Instructions for operating systems and applications are located in a functional form on non-transitory memory for execution by the processor to perform one or more of the processes described herein. These instructions in the different embodiments may be embodied on different physical or tangible computer-readable media, such as memory or another memory, such as a computer-readable media, which may include, without limitation, a flash drive, CD-ROM, thumb drive, floppy disk, etc. Further, instructions are located in a functional form on non-transitory computer-readable media, which may include, without limitation, a flash drive, CD-ROM, thumb drive, floppy disk, etc. Computer-readable media is selectively insertable and/or removable from computing device 8 to permit access to and/or execution by the processor. In one example, computer-readable media includes an optical or magnetic disc that is inserted or placed into a CD/DVD drive or other device associated with memory and/or the processor. In some instances, computer-readable media may not be removable.

In the exemplary embodiment, effector 104 is coupled about conduit 102 to enable the parameters of fluid flowing through conduit 102 to be selectively controlled. Sensors 130 are coupled to conduit 102 to monitor selected parameters of the fluid flowing through conduit 102. Such parameters may include, but are not limited to only including, a temperature and a rate of flow. In the exemplary embodiment, effector 104 is a resistive element. Alternatively, effector 104 may be any device that heats, cools, changes, and/or maintains and/or that functions as described herein. System 100 is configured to control the temperature of fluid with technology including, but not limited to, an ultrasonic, ultraviolet (e.g., UVC), IR, RF, microwave, pump, heat pump, heat sink, convection, and/or conduction device.

In one embodiment, effector 102 includes a plurality of interior channels (not shown) that facilitate heat transfer from or to fluid flowing therethrough. System 100 may be configured to selectively adjust and maintain any parameter. Effector 104 may be coupled inline (i.e., in series flow communication) with conduit 102, or may be coupled in parallel with conduit 102. Moreover effector 4 may be permantly to conduit 102 or alternatively may be releasably coupled thereto. Conduit 102 may be medical grade and/or may have a disposable and/or sterile insert that may be coupled to conduit 102. Any portion or all of system 100 may be sealed, autoclavable, and/or disposable.

During use, fluid is channeled through conduit 130 to a patient and/or to a surrounding surgical area of a patient undergoing a surgical procedure. In the exemplary embodiment, a target or desired parameter of the fluid is selected using an input device, such as device 112. In one embodiment, an acceptable operating range is selected for the parameter being monitored. As fluid is channeled through conduit 102, sensor 130 monitors the parameters of the fluid (e.g., temperature and flow rate). If an operating parameter falls outside of the predetermined or target parameter effector 4 is activated to change the parameter. For example, if the fluid channeled through conduit 102 is at 45° and the target fluid temperature is 60°, effector 104 is activated to heat the fluid to the desired temperature. Conversely, if fluid channeled through conduit 102 is at 60° and the target fluid temperature is 45°, effector 104 is activated to dissipate the heat, and thus, reduce the temperature of the fluid to the desired temperature. Similarly, other parameters (e.g., flow rate) can be maintained.

In one embodiment, at least one sensor 30 is located at or near the patient or within the surgical area of a patient undergoing a surgical procedure. In such an embodiment, operating parameters transmitted from each sensor 30 are received by system 100 and are compared to measured parameters of the fluid channeled through conduit 102. Based on the comparison, controller 8 may control operation of effector 104 to facilitate the adjusting or maintaining of the operating parameters. For example, the temperature of a patient's body may be compared with the temperature of the fluid entering and/or exiting effector 4 en route to the patient's body. Such a comparison may cause controller 108 to activate to facilitate controlling the temperature of the patient's body.

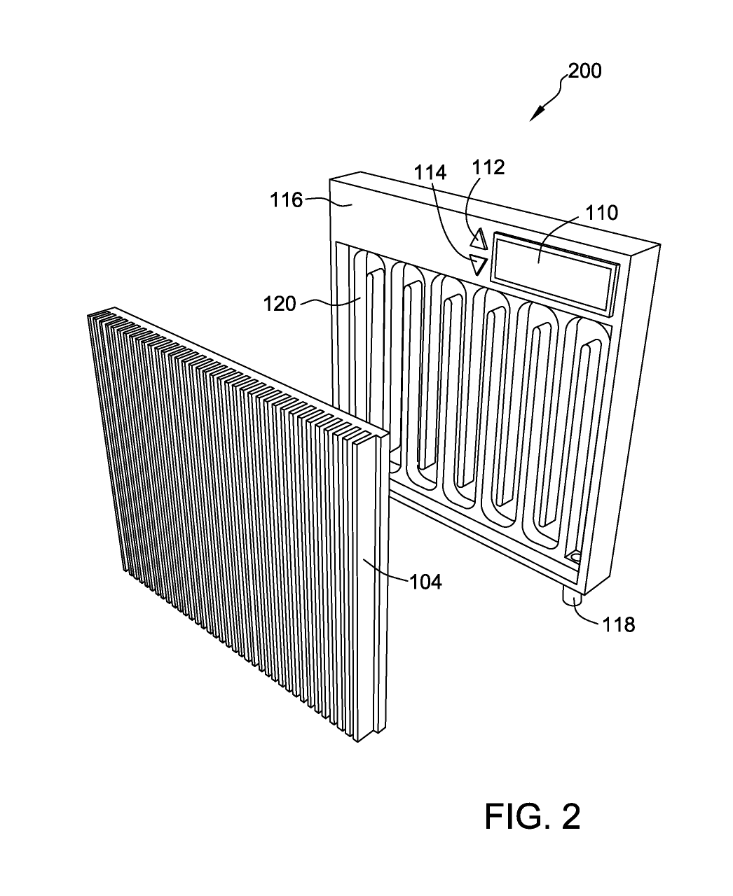

FIG. 2 is a schematic view an alternative environment management system 200 that includes a heat pump 104. Similar to system 100 (shown in FIG. 1), system 200 includes an effector 104 and a computing device or controller 108. System 200 also includes a housing 116 that houses controller 108 therein.

In the exemplary embodiment, effector 104 of system 200 includes functions as a heat pump to facilitate controlling a temperature of fluid entering a patient and/or to selectively control a temperature of the patient's body. Moreover, in the exemplary embodiment, conduit 102 includes sensors 130 that are coupled to system 200 via connector 18, that selectively controls fluid flow into and from chamber 120. Chamber 120 may include a plurality of channels (not shown) that enable fluid to be channeled across an inner surface of effector 104, for example, to facilitate heat transfer. System 200 may be disposable, include a removable insert, and/or be configured to be autoclavable. Additional effectors may be coupled within system 200, including being coupled to effector 104 (e.g., function as a heat sink) and/or in chamber 120.

Similar to system 100, system 200 enables fluid that is channeled through conduit 102 to a patient's body portion or to a surgical area of a patient undergoing a surgical procedure, to be maintained at a predetermined or selected parameter.

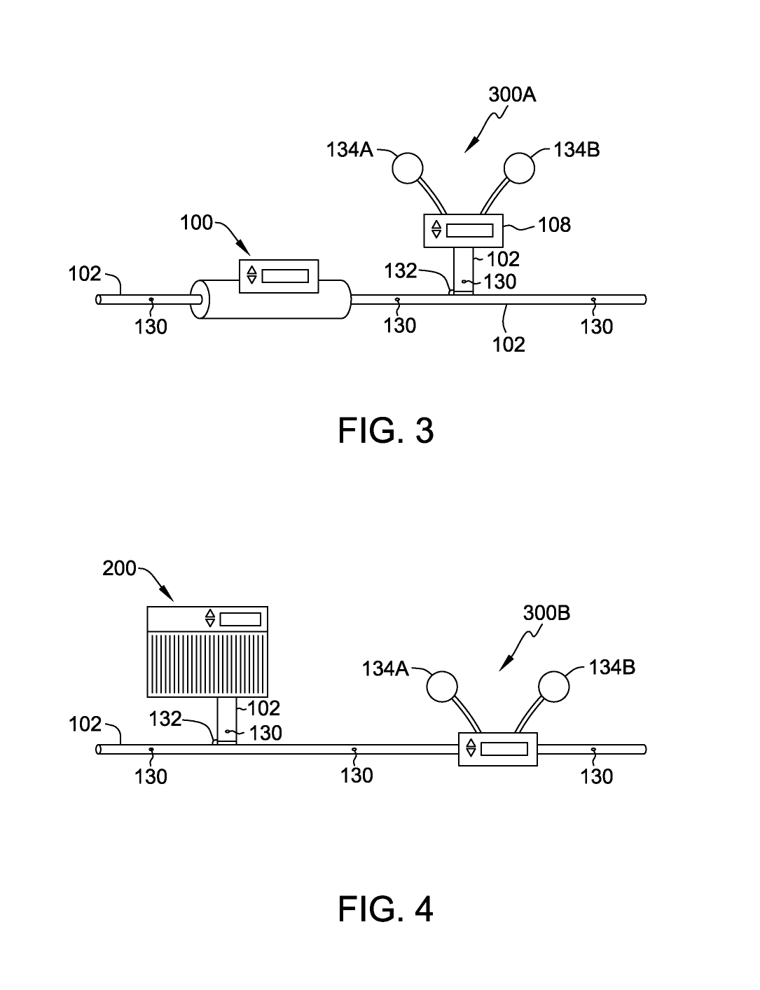

FIG. 3 is a schematic view of an environment management system 300A with environment management system 100 shown in FIG. 1 and FIG. 4 is a schematic view of an environment management system 300B with environment management system 200 shown in FIG. 2. Each of systems 300A and 300B include a controller 108 coupled to reservoirs 134A and 134B. In the exemplary embodiment, reservoirs 134A and 134B are used to adjust and/or maintain pH of fluid flowing to a patient or surgical area. Additionally, reservoirs 134A and 134B can be used to deliver therapeutic substances and/or drugs to a patient or surgical area. In the exemplary embodiment, systems 300A and 300B include sensors 30 communicatively coupled to controller 108 and placed within a patient.

During use, the pH of fluid flowing into a patient's body may be controlled by a gas, liquid, or powder. In one embodiment, reservoir 134A may contain a basic substance, for example, to increase the pH of a fluid and/or portion of a body (e.g., tissue). Reservoir 134B may contain an acidic substance, for example, to lower the pH of a fluid and/or portion of a body (e.g., tissue). By way of example and not limitation, a portion of a patient's body or tissue may need to be made more acidic to promote healing and/or decrease pain. In such an embodiment, carbon dioxide (e.g., liquid carbon dioxide) is delivered to the tissue. As the carbon dioxide is released, carbonic acid would be created which could make the pH of the tissue more acidic.

The pH level of fluid and/or a portion of a patient's body may be automatically or manually controlled using controller 108 with information from sensors 30. Sensors 30 positioned locally in a patient's body can detect the pH level and provide such information to controller 108 to selectively delivery a pH-changing agent. The pH level can be variable selected such that the pH level is changed in accordance therewith.

In an exemplary embodiment, systems 300A and 300B include a pump (not shown) that is configured to control local regulation of pH. In such an embodiment, agents that are configured to alter pH can be placed in the pump which may be externally or internally controlled. Additionally, the pump may be inserted within a patient's body. The can include pH controlling agents, including but not limited to only including, calcium carbonate, calcium sulfate, sodium chloride, and potassium chloride. Calcium based compounds may be used because they are easily metabolized by the body and can help with issues of osteoporosis. Some salts, because of their ability to bind to proteins, may also be efficacious with pH control. Certain salts that are released may have an affinity to bind to proteins and affect the local microclimate. The pump can include valves which release the agents into a patient's body such as into a patient's circulatory system. In one embodiment, the pump can have an osmotic membrane covering. The pH control system could also be ionic anionic.

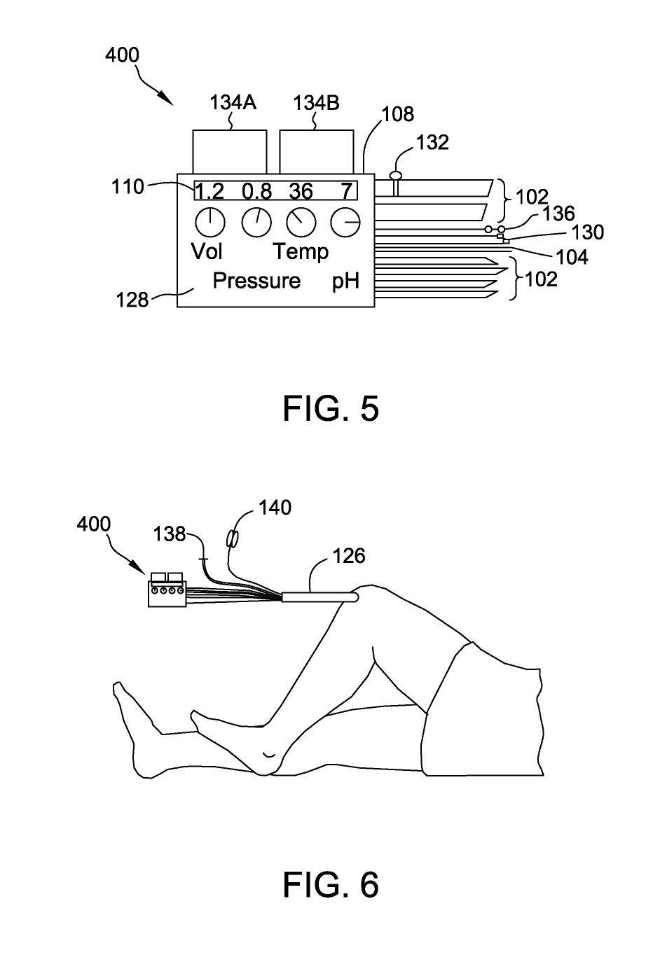

FIG. 5 is an exemplary environment management system 400 that may be used to adjust and/or control one or more parameters of a body undergoing a surgical procedure. In the exemplary embodiment, similar to system 100 (shown in FIG. 1) assembly 400 may include conduits 102, effectors 104 and 132, controller 108, and display 110. System 400 also includes at least one pump 128, sensors 130, at least one reservoir 34A or 34B, and/or magnets 136. Sensors 130 may be temperature sensors, pH sensors, moisture sensors, oxygen sensors, carbon dioxide sensors, or any other sensor that measures environment characteristics or parameters as described herein. Magnets 136 may be earth magnets or electromagnets. Sensors 130, pumps 128, effectors 104 and 132, reservoirs 34A and 34B, and magnets 136 are controlled by controller 108, for example, based on predetermined measurements and/or may be controlled or manually via a remote control, a computing device, or a smartphone.

Sensors 30 and/or magnets 36 may be coupled to the patient's tissue in or around an incision or surgical area, temporarily positioned within a patient, or adjacent to an entry area, such as coupled to a trocar 126. Sensors 130 and magnets 136 may be connected to controller 108 via wires or may be wirelessly coupled to controller 108. Fluids, such as saline, water, plasma, and/or other biocompatible fluids may be to trocar 126 through conduit 102 via pump 128. Alternatively, a gas may be supplied into trocar 126. Effector 132 is used to selectively vary the temperature of the fluid during the surgical procedure. Based on signals transmitted from the sensors 130, and/or on a user's direction, pharmaceutical or therapeutic agents stored in the reservoirs 134 may be selectively released into the fluid stream. System 400 enables the surgical environment of the patient and/or the patient to be more effectively controlled. A humidity level may also be controlled using a valve 132, that is controlled by a controller 108 and/or by the user.

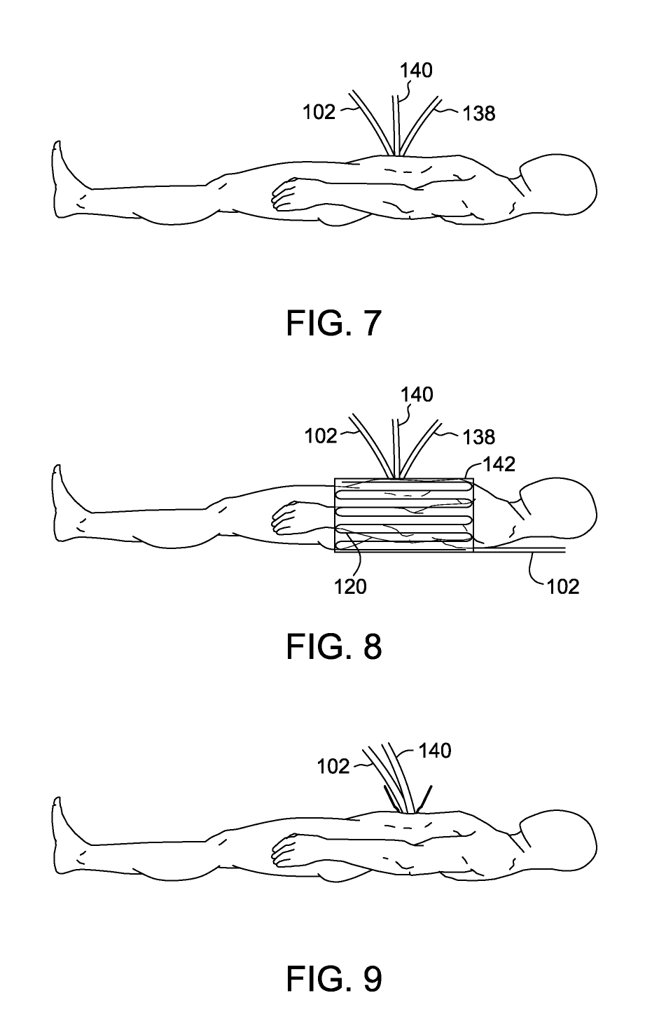

Embodiments may be configured for minimally invasive surgery. For example, FIG. 6 is a schematic of an exemplary environment management system 400 including controller 108 (shown in FIG. 5) and configured for arthroscopic surgery, and FIG. 7 is a schematic of an exemplary environment management system configured for laparoscopic surgery. Embodiments may include a hollow structure, such as a trocar or cannula 126 (e.g., expandable cannula) configured to utilize a minimally invasive incision. In one embodiment, trocar 126 may be remotely and/or magnetically positioned. Trocar 126 may introduce, for example, conduits 102, port 138, and/or port 140. Port 138 may be configured for introduction of medical implants, devices, and/or instruments. Port 140 may be configured for suction and/or injection of fluids and/or therapeutic substances.

Referring to FIG. 8, blanket 42 may include one or more conduits 2 and/or channels, which may be configured to receive fluid. Blanket 42 may be configured to wrap around a portion of the body and/or a surgical area. A parameter (i.e. temperature) of the fluid may be controlled to adjust and/or maintain a parameter of a portion of the body.

With reference to FIG. 9, embodiments may be utilized for an open procedure. Conduit 2 may be configured to adjust and/or maintain a parameter of a fluid and/or portion of the body. Port 40 may be configured for suction and/or injection of fluids and/or therapeutic substances.

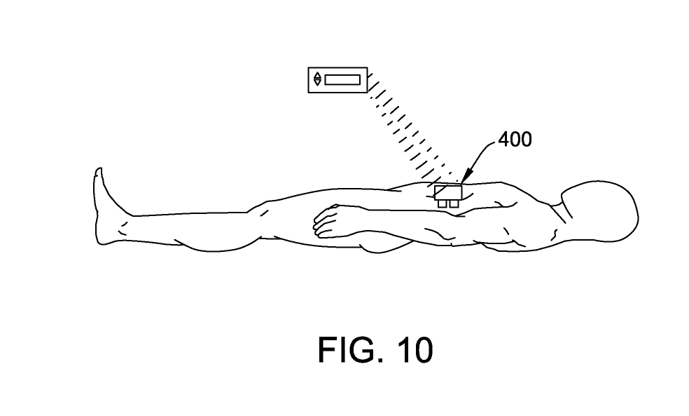

Referring to FIG. 10, embodiments may be implanted in or on a portion of a body and/or utilize wired or wireless control. Wireless may include infrared (IR), radiofrequency (RF), Bluetooth, WiFi, radiowave electromagnetic, and/or microwave transmission. This may allow adjustment of a parameter inside the body from outside the body to optimize a local environment in the body. The adjustment may be based on the local environment, a preoperative plan, and/or intraoperative modeling. Embodiments may be implanted through a trocar or cannula (i.e. expandable cannula), and/or through any percutaneous surgical approach. Embodiments may be positioned by the controller and/or effector.

Embodiments may include one or more sensors, for example, a temperature sensor, pH level sensor, moisture sensor, oxygen sensor, carbon dioxide sensor, or any other sensor to measure environmental characteristics. Also, an electronic processor may also instruct a heating/cooling effector to decrease, increase, or change the temperature of the body region. Controlling the environment may be performed automatically by microprocessors based on preset parameter levels and input signals from the sensors.

The environment may alternatively, or additionally, be controlled by a user via direct or remote control by using a computing device or controller 108. The user may use infrared (IR), radiofrequency (RF), Bluetooth, WiFi, magnetic field, electrical field, radiowave electromagnetic, and/or microwave transmission, for example, to transmit instructions to the microprocessor.

A user, sensor, and/or microprocessor may determine whether the measured parameters are appropriate for the selected body region. If not, the levels may be adjusted. Continuous monitoring of the environment creates a feedback loop so that the environment characteristics may be selectively controlled, manually or automatically. Embodiments may be configured with one or more channels. The sensors and/or effectors for heating and/or cooling may be positioned relative to the channels. The channels may also be configured for delivery of fluids (i.e. gases, liquids, and gels) and solids. Therapeutic agents may be delivered via the channels. Adjustment of pH and temperature may optimize surgery and tissue healing, and minimize postoperative pain.

The above-described embodiments of a method and system for use in controlling an environment of a patient undergoing a surgical procedure. The embodiments disclosed herein facilitate monitoring parameters to change or maintain the parameters at a predetermined level or range. Such parameters can include temperature and/or pH levels in fluids entering a patient's body and/or in a patient's body itself. Maintaining parameters at predetermined levels can affect a patient, the success of a surgical for example, by aiding the body's natural ability to healing, to fight infections, and/or to resist or kill a foreign object.

It should be appreciated that one or more aspects of the present disclosure transform a general-purpose computing device or controller into a special-purpose computing device when configured to perform the functions, methods, and/or processes described herein.

As used herein, an element or step recited in the singular and proceeded with the word “a” or “an” should be understood as not excluding plural elements or steps unless such exclusion is explicitly recited. Furthermore, references to “one embodiment” of the present invention or the “exemplary embodiment” are not intended to be interpreted as excluding the existence of additional embodiments that also incorporate the recited features.

This written description uses examples to disclose various embodiments, which include the best mode, to enable any person skilled in the art to practice those embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system for controlling an environment of a patient undergoing a surgical procedure, said system comprising:
a conduit configured to channel fluids towards the patient;
a sensor configured to measure at least one operating parameter of the fluid being channeled to the patient;
an effector configured to change the operating parameter of the fluid; and
a controller configured to selectively activate said effector based on a signal received from said sensor.

2. The system of claim 1, further comprising a sensor configured to monitor a parameter of the patient.

3. The system of claim 1, wherein said effector includes at least one reservoir configured to hold a first fluid therein.

4. The system of claim 1, wherein said effector includes a a first reservoir for holding a first fluid therein and a second reservoir for holding a second fluid therein.

5. The system of claim 1, wherein said effector includes at least one of a resistive heating element and a heat pump.

6. The system of claim 1, wherein at least a portion of said conduit extends through at least a portion of said effector.

7. The system of claim 1, wherein said effector is releasably coupled to said conduit.

8. A system for use in controlling an environment of a patient, said system comprising:
a conduit configured to channel fluid towards the patient;
a sensor configured to measure an operating parameter of the fluid being channeled to the patient;
a first reservoir configured to selectively release a first substance into said conduit to facilitate increasing the operating parameter of the fluid;
a second reservoir configured to selectively release a second substance into said conduit to facilitate decreasing the operating parameter of the fluid; and
a controller configured to control release of the first and second substances from said first and second reservoirs based on a signal received from said sensor.

9. The system of claim 8, further comprising a body sensor configured to:
measure a parameter of the patient; and
transmit a signal representing the body parameter to said controller.

10. The system of claim 8, wherein said sensor is configured to measure a pH of the fluid being channeled to the patient.

11. The system of claim 8, wherein said first reservoir retains a basic substance therein and said second reservoir retains an acidic substance therein.

12. The system of claim 8, further comprising a resistive heating element coupled to said conduit.

13. The system of claim 8, further comprising a heat pump coupled to said conduit.

14. The system of claim 8, wherein at least a portion of said conduit extends through at least a portion of an effector coupled to said controller.

15. A system for use in controlling an environment of a patient, said system comprising:
a fluid sensor configured to measure a fluid parameter of a fluid channeled towards a patient;
a body sensor configured to measure a body parameter of the patient;
a controller configured to:
compare at least one of the fluid parameter and body parameter to a predefined target parameter; and
change the fluid parameter based on the comparison; and
an effector configured to change the fluid parameter in response to the signal from said controller.

16. The system of claim 15, wherein said effector comprises at least one reservoir.

17. The system of claim 15, wherein said effector is configured to retains at least one of a basic and an acidic substance.

18. The system of claim 15, wherein said effector comprises a resistive heating element.

19. The system of claim 15, wherein said effector comprises a heat pump.

20. The system of claim 15, wherein said heat pump extends through at least a portion of said effector.
